SEQUENCE LISTING (14 sequences)

SEQ ID NO: 1 - protein, 147 aa, Artificial Sequence
  source: /note=“Description of Artificial Sequence: Synthetic polypeptide”
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 2 - protein, 91 aa, Artificial Sequence
  source: /note=“Description of Artificial Sequence: Synthetic polypeptide”
MKKKNSKKSNRTDSKRGDSNGSKSRTACKRCRKKKCDSCKRCAKVCVSDATGKDVRSYVDRAVMMRVKYGVDTKRGNATSDDDKKYSSVSS

SEQ ID NO: 3 - protein, 142 aa, Artificial Sequence
  source: /note=“Description of Artificial Sequence: Synthetic polypeptide”
MKSRTACKRCRLKKIKCDQEFPSCKRCAKLEVPCYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 4 - protein, 143 aa, Artificial Sequence
  source: /note=“Description of Artificial Sequence: Synthetic polypeptide”
MKSRTACKRCRLKKIKCDQEFPSCKRCAKLEVPCVSSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 5 - protein, 146 aa, Artificial Sequence
  source: /note=“Description of Artificial Sequence: Synthetic polypeptide”
MNKKSSEVMHQACDACRKKKWKCSKTVPTCTNCLKYNLDCVYSPQVVRTPLTRAHLTEMENRVAELEQFLKELFPVWDIDRLLQQKDTYRIRELLTMGSTNTVPGLASNNIDSSLEQPVAFGTAQPAQSLSTDPAVQSQAYPMQPV

SEQ ID NO: 6 - protein, 150 aa, Artificial Sequence
  source: /note=“Description of Artificial Sequence: Synthetic polypeptide”
MNKKSSEVMHQACVECRQQKSKCDAHERAPEPCTKCAKKNVPCIVYSPQVVRTPLTRAHLTEMENRVAELEQFLKELFPVWDIDRLLQQKDTYRIRELLTMGSTNTVPGLASNNIDSSLEQPVAFGTAQPAQSLSTDPAVQSQAYPMQPV

SEQ ID NO: 7 - protein, 146 aa, Artificial Sequence
  source: /note=“Description of Artificial Sequence: Synthetic polypeptide”
MNKKSSEVMHQACKRCRLKKIKCDQEFPSCKRCLKYNLDCVYSPQVVRTPLTRAHLTEMENRVAELEQFLKELFPVWDIDRLLQQKDTYRIRELLTMGSTNTVPGLASNNIDSSLEQPVAFGTAQPAQSLSTDPAVQSQAYPMQPV

SEQ ID NO: 8 - protein, 146 aa, Artificial Sequence
  source: /note=“Description of Artificial Sequence: Synthetic polypeptide”
MNKKSSEVMHQACKRCRLKKIKCDQEFPSCKRCAKLEVPCVYSPQVVRTPLTRAHLTEMENRVAELEQFLKELFPVWDIDRLLQQKDTYRIRELLTMGSTNTVPGLASNNIDSSLEQPVAFGTAQPAQSLSTDPAVQSQAYPMQPV

SEQ ID NO: 9 - skipped

SEQ ID NO: 10 - protein, 690 aa, Artificial Sequence
  source: /note=“Description of Artificial Sequence: Synthetic polypeptide”
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSASGSGRAGKPIPNPLLGLDSTDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 11 - protein, 75 aa, Artificial Sequence
  source: /note=“Description of Artificial Sequence: Synthetic polypeptide”
RAGKPIPNPLLGLDSTDALDDFDLDMLGSDALDDFDLDMLGSDALDDFDLDMLGSDALDDFDLDMLGSPKKKRKV

SEQ ID NO: 12 - protein, 265 aa, Artificial Sequence
  source: /note=“Description of Artificial Sequence: Synthetic polypeptide”
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 13 - protein, 190 aa, Artificial Sequence
  source: /note=“Description of Artificial Sequence: Synthetic polypeptide”
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 14 - protein, 538 aa, Artificial Sequence
  source: /note=“Description of Artificial Sequence: Synthetic polypeptide”
RAGKPIPNPLLGLDSTDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

(19) United States
(12) Patent Application Publication
(10) Pub. No.: US 2022/0177897 A1
(43) Pub. Date: Jun. 9, 2022

(54) TRANSCRIPTIONAL RELAY SYSTEM

(71) Applicant: Octant, Inc., Emeryville, CA (US)

(72) Inventors: Leon Yen-Lee CHAN, Berkeley, CA (US); Aaron Ross COOPER, Berkeley, CA (US); Henry CHAN, Los Angeles, CA (US)

(21) Appl. No.: 17/532,791

(22) Filed: Nov. 22, 2021

Related U.S. Application Data
(63) Continuation of application No. PCT/US2020/034685, filed on May 27, 2020.
(60) Provisional application No. 62/853,637, filed on May 28, 2019.

(51) Int. Cl. C12N 15/63; C12N 15/85

(52) U.S. Cl. CPC C12N 15/635; C12N 2830/005; C12N 15/85

(57) ABSTRACT

Described herein are transcriptional relay systems useful for reducing background signal in protein expression and reporter assays. These systems utilize a nucleic acid system wherein a promoter sequence controls expression of a synthetic transcription factor that activates transcription of a reporter molecule.

### CROSS REFERENCE

This application claims the benefit of International Application No. PCT/US2020/034685 filed May 27, 2020, which claims the benefit of U.S. Provisional Application No. 62/853,637 filed May 28, 2019, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 30, 2020, is named, 52652_706_301_SL.txt and is 26,977 bytes in size.

### SUMMARY

Described herein are nucleic acids, systems, and methods useful for interrogating cell signaling pathway responses, screening for antagonists or agonists of cell signaling pathways, or discovering novel cell signaling pathways. Previously known methods in the art utilize endogenous response element regulated promoters proximal to nucleic acids encoding reporter molecules. These methods suffer from high degrees of background signal of the reporter molecules due to the “leaky” nature of the endogenous response element binding promoters in cells. Also, these methods suffer from high a coefficient of variation. Finally, such methods suffer from low absolute values of reporter activation resulting in low signal to noise. The nucleic acids and systems of the present disclosure reduce the level of biological variation, increase signal to noise ratio of reporter signal, and reduce background signal by using a non-endogenous synthetic transcription factor, which is highly selective for a synthetic transcription factor binding site. Thus, transcription of the reporter molecule is not initiated by endogenous transcription factors, helping to reduce background signal and increase signal to noise of the reporter. These nucleic acids and systems are useful for screening small-molecule or biologic agonists or antagonists of signaling pathways, such as G-protein coupled receptors, receptor tyrosine kinases, ion channels, and nuclear receptors. In a broad aspect, the system comprises nucleic acid that encode: a) a response element regulated promoter proximal to the 5′ end of a synthetic transcription factor reading frame; and b) a promoter element capable of being bound by the synthetic transcription factor, said promoter element proximal to the 5′ end of a reporter gene reading frame. In this system the reporter gene may comprise a unique molecular identifier (UMI) to allow for multiplexing of a reporter assay.

In one aspect, described herein, is a transcriptional relay system comprising; a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor; and a reporter nucleic acid comprising a synthetic transcription factor promoter nucleotide sequence and a nucleotide sequence encoding a reporter, wherein said synthetic transcription factor promoter nucleotide sequence is 5′ to said nucleotide sequence encoding said reporter, and wherein said synthetic transcription factor promoter nucleotide sequence is able to be bound by said synthetic transcription factor. In certain embodiments, said response element regulated promoter nucleotide sequence comprises a cAMP response element nucleotide sequence, a NFAT transcription factor response element nucleotide sequence, a FOS promoter nucleotide sequence, or a serum response element nucleotide sequence. In certain embodiments, said synthetic transcription factor comprises a DNA binding domain from a first transcription factor and a transcription activating domain from a second transcription factor. In certain embodiments, said DNA binding domain is from Gal4, PPR1, Lac9, or LexA. In certain embodiments, said DNA binding domain comprises an amino acid sequence at least about 90% identical to that set forth in SEQ ID NO: 1. In certain embodiments, said DNA binding domain comprises an amino acid sequence at least about 95% identical to that set forth in SEQ ID NO: 1. In certain embodiments, said DNA binding domain comprises the amino acid sequence set forth in SEQ ID NO: 1. In certain embodiments, said DNA binding domain comprises an amino acid sequence variant of SEQ ID NO: 1. In certain embodiments, said transcription activating domain comprises VP64, p65, and Rta. In certain embodiments, said transcription activating domain comprises an amino acid sequence at least about 90% identical to that set forth in SEQ ID NO: 14. In certain embodiments, said transcription activating domain comprises an amino acid sequence at least about 95% identical to that set forth in SEQ ID NO: 14. In certain embodiments, said transcription activating domain comprises the amino acid sequence set forth in SEQ ID NO: 14. In certain embodiments, said transcription activating domain comprises an amino acid sequence variant of SEQ ID NO: 14, wherein said sequence variant increases or decreases transcriptional activation. In certain embodiments, said synthetic transcription factor comprises the amino acid sequence variant set forth in SEQ ID NO: 10. In certain embodiments, said synthetic transcription factor comprises a polypeptide sequence that destabilizes said synthetic transcription factor. In certain embodiments, said polypeptide sequence that destabilizes said synthetic transcription factor comprises a PEST or a CL1 polypeptide sequence. In certain embodiments, said synthetic transcription factor promoter nucleotide sequence comprises a nucleotide sequence able to be bound by Gal4, PPR1, Lac9, or LexA. In certain embodiments, reporter comprises a fluorescent protein, a luciferase protein, a beta-galactosidase, a beta-glucuronidase, a chloramphenicol acetyltransferase, a secreted placental alkaline phosphatase, or a unique molecular identifier. In certain embodiments, said reporter comprises a fluorescent protein, a luciferase protein, a beta-galactosidase, a beta-glucuronidase, a chloramphenicol acetyltransferase, or a secreted placental alkaline phosphatase, and a UMI. In certain embodiments, said unique molecular identifier is unique to a test polypeptide, wherein said test polypeptide is encoded by said reporter nucleic acid. In certain embodiments, said transcription factor nucleic acid comprises a nucleotide sequence proximal to said response element regulated promoter nucleotide sequence that can be bound by transcriptional repressors. In certain embodiments, said transcription factor nucleic acid comprises a nucleotide sequence proximal to said response element regulated promoter nucleotide sequence that extends the 5′ untranslated region of an mRNA encoded by said nucleotide sequence encoding a synthetic transcription factor. In certain embodiments, wherein said 5′ untranslated region of an mRNA encoded by said nucleotide sequence encoding a synthetic transcription factor comprises one or more sequences that reduce translation of said synthetic transcription factor. In certain embodiments, said transcription factor nucleic acid and said reporter nucleic acid are components of a single nucleic acid. In certain embodiments, as described herein, is a cell comprising said relay system. In certain embodiments, said cell comprises a eukaryotic cell. In certain embodiments, said cell comprises a mammalian cell. In certain embodiments, the transcription factor nucleic acid, the reporter nucleic acid, or both the transcription factor nucleic acid and the reporter nucleic acid are integrated as a single copy into the genome of the cell. In certain embodiments, as described herein, is a cell population comprising said relay system. In certain embodiments, said cell population comprises a population of eukaryotic cells. In certain embodiments, said cell population comprises a population of mammalian cells. In certain embodiments, the cell or cell population comprises high basal reporter activity. In certain embodiments, the cell or cell population comprises wherein the high basal reporter activity is at least about 30× greater than background, wherein background is the level of reporter activity observed for a parental cell or cell line that does not comprise the reporter. In certain embodiments, the cell or cell population comprises a low biological coefficient of variance for reporter activity. In certain embodiments, the cell or cell population comprises wherein the low biological coefficient of variance for reporter activity is below about 0.5.

In certain embodiments, as described herein, is a method for testing an effect of a test agent on the activity of a response element regulated promoter comprising contacting a cell or a population of cells with said test substance. In certain embodiments, said test agent is a chemical.

### BRIEF DESCRIPTION OF THE DRAWINGS

FIG. 1A depicts a schematic of a transcriptional relay system, showing a transcription factor nucleic acid (left) and a reporter nucleic acid (right).

FIG. 1B depicts a nucleic acid sequence encoding a reporter wherein said reporter comprises a unique RNA sequence.

FIG. 2 shows reporter output for cells carrying a singly integrated CRE-luciferase (grey) and cells carrying a single integrated UAS-luciferase along with multiple copies of semi-randomly integrated CRE-Gal4-VPR (black).

FIG. 3 shows the coefficient of variation for each sample depicted in FIG. 2, which were run in triplicate.

FIG. 4 shows the effect of a destabilizing sequence tag (degron tag) on a Gal4-VPR promoter nucleotide sequence on the fold induction of a transcriptional relay system.

FIG. 5 shows cell libraries generated from NFAT-relay isoclonal cell lines. Cell lines were screened for their ability to detect NFAT-relay reporter activity for Gq coupled GPCRs with positive control compounds. Receptor-compound combinations that generated signals with lower than 0.001 false discovery rate (FDR) or with a max_Q of greater than 3 were deemed as significant hits. Libraries cb29 and cb37, generated the most significant hits in this screen.

FIG. 6 shows variance vs. basal activity of isoclonal cell lines that were used to generate the cell libraries.

### DETAILED DESCRIPTION

In one aspect, described herein, is a transcriptional relay system comprising; (a) a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor; and (b) a reporter nucleic acid comprising a synthetic transcription factor promoter nucleotide sequence and a nucleotide sequence encoding a reporter, wherein said synthetic transcription factor promoter nucleotide sequence is 5′ to said nucleotide sequence encoding said reporter, and wherein said synthetic transcription factor promoter nucleotide sequence is able to be bound by said synthetic transcription factor.

In another aspect, described herein, is a method to assay an effect of a test substance on the activity of a response element regulated promoter comprising; (a) contacting a cell with a test substance, said cell comprising (i) a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor; and (ii) a reporter nucleic acid comprising a synthetic transcription factor promoter nucleotide sequence and a nucleotide sequence encoding a reporter, wherein said synthetic transcription factor promoter nucleotide sequence is 5′ to said nucleotide sequence encoding said reporter, and wherein said synthetic transcription factor promoter nucleotide sequence is able to be bound by said synthetic transcription factor; and (b) conducting at least one assay that measures transcription of said reporter.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10%.

The terms “polypeptide” and “protein” are used interchangeably to refer to a polymer of amino acid residues, and are not limited to a minimum length. Polypeptides, including the provided polypeptide chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “identity,” “identical,” or “percent identical” when used herein to describe to a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent identity of sequences can be determined using the most recent version of BLAST, as of the filing date of this application.

The polypeptides of the systems described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements for integration.

As used herein the term “transfection” or “transfected” refers to methods that intentionally introduce an exogenous nucleic acid into a cell through a process commonly used in laboratories. Transfection can be effected by, for example, lipofection, calcium phosphate precipitation, viral transduction, or electroporation. Transfection can be either transient or stable.

As used herein the term “transfection efficiency” refers to the extent or degree to which a population of cells has incorporated an exogenous nucleic acid. Transfection efficiency can be measured as a percentage (%) of cells in a given population that have incorporated an exogenous nucleic acid compared to the total population of cells in a system. Transfection efficiency can be measured in both transiently and stably transfected cells.

As used herein, the term “biologically activating polypeptide” refers to a polypeptide expressed by a cell that modulates gene expression. The biologically activating polypeptide may modulate gene expression directly, through signaling via one or more intermediary molecules or polypeptides, in response to a stimuli, or through any other mechanism. A biologically activating polypeptide may be a transmembrane polypeptide (such as a receptor or a channel protein), an intracellular polypeptide (such as signal transduction intermediaries), an extracellular polypeptide, or a secreted polypeptide.

As used herein “reporter activity” refers to the empirical readout from the reporter. For example, a luciferase reporter will have a luminescent readout when incubated with an appropriate substrate. Other reporters like a fluorescent protein may not require a substrate but can be measured via microscopy or a fluorescence plate reader for example.

### System Overview

The systems, nucleic acids, and methods described herein are useful to screen for the presence and/or level of activation of a response element binding promoter. The nucleic acids, systems, and method described herein allow for activation of transcription with lower levels of background signal than traditional reporter systems. In certain embodiments, a response element binding promoter is activated at the end of a cell signaling cascade. In certain embodiments, the presence of a response element binding promoter can be measured before and after an external stimulus such as a physical or chemical stimulus, or compared to control conditions run in parallel. The chemical stimulus can be an agonistic or antagonistic small molecule or biologic molecule. In certain embodiments, the system is useful for screening for pharmaceutical discovery purposes. The system minimally comprises nucleic acid(s) comprising a response element regulated promoter, a synthetic transcription factor promoter, a synthetic transcription factor, and a reporter. The response element regulated promoter is positioned 5′ to the synthetic transcription factor and activates transcription of the synthetic transcription factor when the response element binding promoter is present. Upon translation, the synthetic transcription factor may then bind to the synthetic transcription factor promoter, which is located 5′ to the nucleic acid sequence encoding the reporter. While bound, the synthetic transcription factor promoter activates transcription of the nucleic acid sequence encoding the reporter. In certain embodiments, the reporter is a polypeptide. In certain embodiments, the reporter is a UMI. Additional optional features of the system include a nucleotide sequence proximal to the response element regulated promoter nucleotide sequence that can be bound by transcriptional repressors. In certain embodiments, the nucleotide sequence proximal to the response element regulated promoter nucleotide sequence extends the 5′ untranslated region of the mRNA encoded by the nucleotide sequence encoding the synthetic transcription factor. In certain embodiments, the 5′ untranslated region of the mRNA encoded by the nucleotide sequences encoding the synthetic transcription factor has one or more sequences that reduce translation of the synthetic transcription factor.

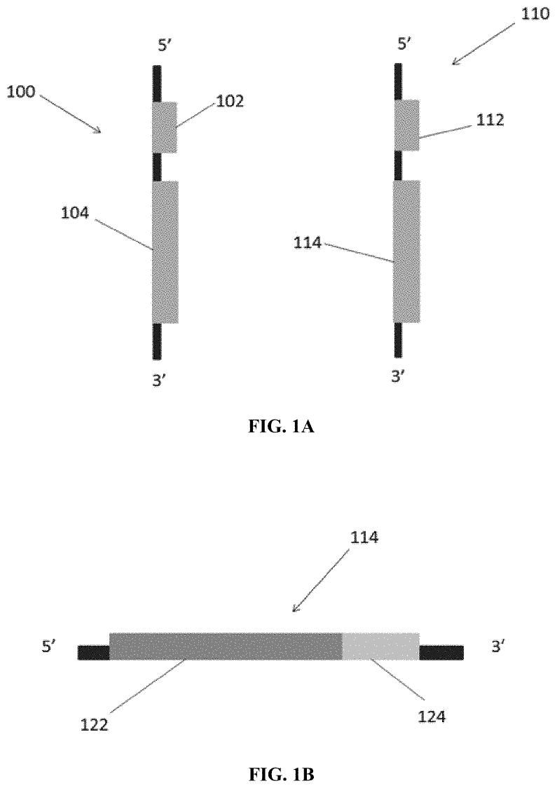

One non-limiting embodiment of the present invention is shown in FIG. 1A. A transcription factor nucleic acid 100 is shown at left. Present on the transcription factor nucleic acid 100 is a response element regulated promoter nucleic acid 102 in the 5′ position of a nucleotide sequence encoding a synthetic transcription factor 104. At right is a reporter nucleic acid 110, which contains a synthetic transcription factor promoter nucleotide sequence 112, which is 5′ of a nucleotide sequence encoding a reporter 114. In certain embodiments, the transcription factor nucleic acid and the reporter nucleic acid are present on separate nucleic acid molecules, for example separate plasmids or viral vectors. In certain embodiments, the transcription factor nucleic acid and the reporter nucleic acid are linear. In certain embodiments, the transcription factor nucleic acid and the reporter nucleic acid are present on the same nucleic acid, which may be a plasmid, viral vector, linear, or any other configuration.

One non-limiting embodiment of a nucleotide sequence encoding a reporter is shown in FIG. 1B. A nucleotide sequence encoding a reporter 114 comprises a nucleic acid sequence encoding a reporter polypeptide 122 as well as a nucleic acid sequence encoding a UMI 124. Sequence 124 is also known as a unique molecular identifier (UMI). The UMI can identify a particular biologically activating polypeptide that results in activation of the response element regulated promoter nucleic acid at 102. By way of non-limiting example, the biologically activating polypeptide can comprise a particular G-coupled protein receptor, of which there are several hundred known. Thus, the UMI element allows for easy and rapid interrogation of the signaling of several different biologically activating polypeptides in multiplex format. Additionally, the relay system provided reduces background signaling through a response element regulated promoter. This allows for more accurate quantification, and reduces the number of false positive test compounds in any multiplex screening for compounds that may activate a biologically activating polypeptide. In certain embodiments, the nucleic acid sequence encoding a reporter polypeptide is absent. In certain embodiments, the nucleic acid sequence encoding a UMI is absent. In certain embodiments, the nucleic acid sequence encoding a UMI is 5′ of the nucleic acid sequence encoding the reporter polypeptide. In certain embodiments, the nucleic acid sequence encoding the reporter polypeptide is 5′ of the nucleic acid sequence encoding a UMI.

In certain embodiments, a nucleic acid encoding a reporter encodes a reporter polypeptide. In certain embodiments, said reporter polypeptide is capable of being detected directly. In certain embodiments, said reporter polypeptide produces a detectable signal upon the protein's enzymatic activity to a substrate. In certain embodiments, detection of a reporter polypeptide can be accomplished quantitatively. In certain embodiments, said reporter polypeptide comprises a luciferase protein, a beta-galactosidase, a beta-glucuronidase, a chloramphenicol acetyltransferase, a secreted placental alkaline phosphatase, or combinations thereof. In certain embodiments wherein said reporter polypeptide is a luciferase protein, non-limiting examples of substrates include firefly luciferin, latia luciferin, bacterial luciferin, coelenterazine, dinoflagellate luciferin, vargulin, and 3-hydroxy hispidin.

In certain embodiments, a nucleic acid encoding a reporter encodes a UMI. Said UMI comprises a short sequence of nucleotides that is unique to the nucleic acid. Said UMI may be 8, 9, 10, 11, 12, 13, 14, 15, 16, 17, 18, 19, 20, or more nucleotides in length. Said UMI is capable of being detected in any suitable way that allows sequence determination of said UMI, such as by next-generation sequencing methods. Methods of detecting said UMI may be quantitative, and include next-generation sequencing methods.

In certain embodiments, described herein, is a method of deploying a system comprising nucleic acid(s) encoding a transcription factor nucleic acid and a reporter nucleic acid for use in drug discovery. In certain embodiments, the method comprises contacting the nucleic acid(s) with a cell or population of cells under conditions sufficient for the nucleic acid(s) to be internalized and expressed by the cell (e.g., transfected); contacting the cell with a physical or chemical stimulus; and determining activation of the reporter element by one or more assays. In certain embodiments, the method comprises contacting a cell or population of cells comprising nucleic acid(s) encoding a transcription factor nucleic acid and a reporter nucleic acid; and determining activation of the reporter element by one or more assays.

Response Element Regulated Promoters

Response elements are short sequences of DNA within a gene promoter region that are able to bind specific transcription factors and regulate transcription of genes. Certain response elements are specific to certain promoters. Some response elements are capable of being bound by endogenous transcription factors. Multiple copies of the same response element can be located in different portions of a nucleotide sequence, activating different genes in response to the same stimuli. Non-limiting examples of response elements that can be incorporated in to the system described herein include cAMP response element (CRE), B recognition element, AhR-, dioxin- or xenobiotic- responsive element, HIF-responsive elements, hormone response elements, serum response element, retinoic acid response elements, peroxisome proliferator hormone response elements, metal-responsive element, DNA damage response element, IFN-stimulated response elements, ROR-response element, glucocorticoid response element, calcium-response element CaRE1, antioxidant response element, p53 response element, thyroid hormone response element, growth hormone response element, sterol response element, polycomb response elements, and vitamin D response element.

Response element regulated promoter nucleotide sequences are regions of nucleic acids containing one or more response elements that aid in recruiting promoters and other molecules to regulate transcription of genes. Cells contain many response element regulated nucleotide sequences that utilize endogenous proteins to modulate transcription of genes. In situations where an endogenous response element regulated promoter nucleotide sequence directly regulates transcription of a reporter, there exists a high level of background signal due to the presence of endogenous promoters. A system that regulates transcription of a reporter with a transcription factor that is not endogenous to a cell containing said system would have advantages over a system that regulates transcription of a reporter with an endogenous transcription factor. One advantage of such a system would be a lower background production of said reporter.

In certain embodiments, a transcriptional relay system of the present invention comprises a transcription factor nucleic acid comprising a response element regulated promoter nucleotide sequence and a nucleotide sequence encoding a synthetic transcription factor, wherein said response element regulate promoter nucleotide sequence is 5′ to said nucleotide sequence encoding said synthetic transcription factor. Said response element regulated promoter nucleotide sequence acts to control expression of a synthetic transcription factor encoded by said synthetic transcription factor nucleotide sequence. In certain embodiments, said response element regulated promoter nucleotide sequence comprises a cAMP response element nucleotide sequence, a NFAT transcription factor response element nucleotide sequence, a FOS promoter nucleotide sequence, a serum response element nucleotide sequence, or combinations thereof. In certain embodiments, said response element regulated promoter nucleotide sequence comprises a cAMP response element nucleotide sequence. In certain embodiments, said response element regulated promoter nucleotide sequence comprises a NFAT transcription factor response element nucleotide sequence. In certain embodiments, said response element regulated promoter nucleotide sequence comprises a FOS promoter nucleotide sequence. In certain embodiments, said response element regulated promoter nucleotide sequence comprises a serum response element nucleotide sequence. In certain embodiments, said response element regulated promoter nucleotide sequence comprises any combination of a cAMP response element nucleotide sequence, a NFAT transcription factor response element nucleotide sequence, a FOS promoter nucleotide sequence, and/or a serum response element nucleotide sequence.

In certain embodiments, said response element regulated promoter is capable of being bound by a transcription factor. Non-limiting examples of common transcription factors include LexA, Gal4, VP16 (from Herpes Simplex Virus), heat shock factor (HSF), NFAT, CREB, or combinations thereof. The system described herein is compatible with any transcription factor commonly or potentially useable in a reporter assay, or any combination thereof.

In certain embodiments, said response element regulated promoter is bound by an endogenous transcription factor. Endogenous transcription factors are transcription factors which are naturally present in an organism, tissue, or cell. The presence of endogenous transcription factors will depend upon the system in which said transcription relay is present. In certain embodiments, said endogenous transcription factors promote transcription of a synthetic transcription factor at a background rate.

In certain embodiments, said transcription factor nucleic acid comprises a nucleotide sequence proximal to said response element regulated promoter nucleic acid sequence that can be bound by transcriptional repressors. Transcriptional repressors inhibit transcription of distal nucleotide sequences. Non-limiting examples of common transcriptional repressors include TetR, lac repressors, KRAB repressors, and combinations thereof. The system described herein is compatible with any repressor commonly or potentially useable in a reporter assay, or combinations thereof.

In certain embodiments, said transcription factor nucleic acid comprises a nucleotide sequence proximal to said response element regulated promoter nucleotide sequence that extends the 5′ untranslated region of an mRNA encoded by said nucleotide sequence encoding a synthetic transcription factor. In certain embodiments, said 5′ untranslated region of an mRNA encoded by said nucleotide sequence encoding a synthetic transcription factor comprises one or more sequences that reduce translation of said synthetic transcription factor. In certain embodiments, said one or more sequences that reduces translation of said synthetic transcription factor comprises a secondary structure that reduces translation of said synthetic transcription factor. In certain embodiments, said one or more sequences that reduces translation of said synthetic transcription factor comprises a sequence that affects binding by RNA binding proteins. In certain embodiments, said one or more sequences that reduces translation of said synthetic transcription factor comprises an upstream open reading frame.

### Assay Methods

The system described above can be effectively utilized using a variety of methods. The system is useful in methods to interrogate activity of cell signaling pathways, both at a steady-state and in response to a physical or chemical stimulus. When the reporter element comprises a UMI sequence mated to a particular reporter element, the system can be deployed in a multiplexed assay.

In one non-limiting, illustrative example, a plurality of cells are incubated in one well of a multi-well plate. The plurality of cells are transfected with a reporter nucleic acid comprising a synthetic transcription factor promoter nucleotide sequence and a nucleotide sequence encoding a reporter. The cells can already comprise a transcription factor nucleic acid comprising a response element regulated promoter nucleotide sequence and a nucleotide sequence encoding a synthetic transcription factor, or can be transfected with said transcription factor nucleic acid. The transfected cells are then contacted with a chemical stimulus. After a sufficient amount of time to allow for expression of a reporter gene, cell lysates are harvested and activation of said reporter gene quantified. In this example, increased presence of a reporter gene would be indicative of a chemical stimulus causing an increase in the activity of transcription factor(s) that bind(s) said response element regulated promoter. In certain embodiments, said transcription factor(s) that bind(s) said response element regulated promoter has increased activity following a cell-signaling cascade.

In embodiments wherein said reporter gene comprises an enzyme that produces a detectable signal upon interaction with a substrate, standard assays known in the art can be utilized to quantify activation said reporter gene. In embodiments wherein said reporter gene comprises a fluorescent molecule, the activation of said reporter gene can be measured by fluorescence microscopy or a fluorescent plate reader, and may not require cell lysis. Said fluorescent molecules are useful for measuring reporter activation in live cells. In embodiments wherein said reporter gene comprises UMI, mRNA is reverse transcribed, and sequencing of the UMI is performed by next-generation sequencing technology.

In certain embodiments, the assays are carried out in multiwell formats such as 6, 12, 24, 48, 96, or 384-well format. In certain embodiments, each well is supplied with a different test chemical, or the test chemicals are supplied in duplicate, triplicate, or quadruplicate wells. The assay can also comprise one or more positive or a negative control wells.

### Synthetic Transcription Factors

Synthetic transcription factors are artificial proteins capable of targeting and modulating gene expression. Some synthetic transcription factors are chimeric proteins containing domains from multiple different genes. In certain embodiments, synthetic transcription factors comprise a DNA binding domain from one gene and transcriptional regulatory domain from another gene.

In the methods, nucleic acids, and systems described herein a transcriptional activating polypeptide is encoded on a transcription factor nucleic acid. In certain embodiments, said transcription activating polypeptide is a synthetic transcription factor. In certain embodiments, said synthetic transcription factor is a chimeric protein. In certain embodiments, said synthetic transcription factor comprises a DNA binding domain from a first transcription factor. In certain embodiments, said synthetic transcription factor comprises a transcription activating domain from a second transcription factor. In certain embodiments, said first transcription factor is different than said second transcription factor.

In certain embodiments, said synthetic transcription factor has a higher specificity for a synthetic transcription factor promoter nucleotide sequence than any endogenous transcription factor. In certain embodiments, said synthetic transcription factor binds a synthetic transcription factor promoter nucleotide sequence not capable of being bound by an endogenous promoter. In certain embodiments, said synthetic transcription factor results in less background production of a reporter than would occur with use of an endogenous transcription factor.

In certain embodiments, said DNA binding domain is non-endogenous to a cell containing a transcriptional relay system of the present invention. In certain embodiments, said DNA binding domain from a first transcription factor is from Gal4, PPR1, LexA, Lac9, or combinations thereof. In certain embodiments, said DNA binding domain comprises an amino acid sequence set forth in MKLLSSIEQACDICRLKKLKCSKEKPKCAKCLKNNWECRYSPKTKRSPLTRAHLTEVESRLE RLEQLFLLIFPREDLDMILKMDSLQDIKALLTGLFVQDNVNKDAVTDRLASVETDMPLTLRQ HRISATSSSEESSNKGQRQLTVS, SEQ ID NO: 1. In certain embodiments, said DNA binding domain comprises an amino acid sequence set forth in MKKKNSKKSNRTDSKRGDSNGSKSRTACKRCRKKKCDSCKRCAKVCVSDATGKDVRSYV DRAVMMRVKYGVDTKRGNATSDDDKKYSSVSS, SEQ ID NO: 2. In certain embodiments, said DNA binding domain comprises an amino acid sequence set forth in MKSRTACKRCRLKKIKCDQEFPSCKRCAKLEVPCYSPKTKRSPLTRAHLTEVESRLERLEQL FLLIFPREDLDMILKMDSLQDIKALLTGLFVQDNVNKDAVTDRLASVETDMPLTLRQHRISA TSSSEESSNKGQRQLTVS, SEQ ID NO: 3. In certain embodiments, said DNA binding domain comprises an amino acid sequence set forth in MKSRTACKRCRLKKIKCDQEFPSCKRCAKLEVPCVSSPKTKRSPLTRAHLTEVESRLERLEQ LFLLIFPREDLDMILKMDSLQDIKALLTGLFVQDNVNKDAVTDRLASVETDMPLTLRQHRIS ATSSSEESSNKGQRQLTVS, SEQ ID NO: 4. In certain embodiments, said DNA binding domain comprises an amino acid sequence set forth in MNKKSSEVMHQACDACRKKKWKCSKTVPTCTNCLKYNLDCVYSPQVVRTPLTRAHLTEM ENRVAELEQFLKELFPVWDIDRLLQQKDTYRIRELLTMGSTNTVPGLASNNIDSSLEQPVAF GTAQPAQSLSTDPAVQSQAYPMQPV, SEQ ID NO: 5. In certain embodiments, said DNA binding domain comprises an amino acid sequence set forth in MNKKSSEVMHQACVECRQQKSKCDAHERAPEPCTKCAKKNVPCIVYSPQVVRTPLTRAHL TEMENRVAELEQFLKELFPVWDIDRLLQQKDTYRIRELLTMGSTNTVPGLASNNIDSSLEQP VAFGTAQPAQSLSTDPAVQSQAYPMQPV, SEQ ID NO: 6. In certain embodiments, said DNA binding domain comprises an amino acid sequence set forth in MNKKSSEVMHQACKRCRLKKIKCDQEFPSCKRCLKYNLDCVYSPQVVRTPLTRAHLTEME NRVAELEQFLKELFPVWDIDRLLQQKDTYRIRELLTMGSTNTVPGLASNNIDSSLEQPVAFG TAQPAQSLSTDPAVQSQAYPMQPV, SEQ ID NO: 7. In certain embodiments, said DNA binding domain comprises an amino acid sequence set forth in

SEQ ID NO: 8
MNKKSSEVMHQACKRCRLKKIKCDQEFPSCKRCAKLEVPCVYSPQVVRTP
LTRAHLTEMENRVAELEQFLKELFPVWDIDRLLQQKDTYRIRELLTMGST
NTVPGLASNNIDSSLEQPVAFGTAQPAQSLSTDPAVQSQAYPMQPV,.

In certain embodiments, said DNA binding domain comprises an amino acid sequence variant of SEQ ID NO: 1. In certain embodiments, the amino acid sequence variant of SEQ ID NO: 1 is R15W, K23P, K23T, K23W, K23M, K23N, F68R, F68Q, L69P, L70P, Q9E, Q9A, Q9N, R15K, R15A, R15M, K18R, K18A, K18M, K23R, K23A, K23M, or combinations thereof. In certain embodiments, the amino acid sequence variant of SEQ ID NO: 1 is R15W. In certain embodiments, the amino acid sequence variant of SEQ ID NO: 1 is K23P. In certain embodiments, the amino acid sequence variant of SEQ ID NO: 1 is K23T. In certain embodiments, the amino acid sequence variant of SEQ ID NO: 1 is K23W. In certain embodiments, the amino acid sequence variant of SEQ ID NO: 1 is K23M. In certain embodiments, the amino acid sequence variant of SEQ ID NO: 1 is K23N. In certain embodiments, the amino acid sequence variant of SEQ ID NO: 1 is F68R. In certain embodiments, the amino acid sequence variant of SEQ ID NO: 1 is F68Q. In certain embodiments, the amino acid sequence variant of SEQ ID NO: 1 is L69P. In certain embodiments, the amino acid sequence variant of SEQ ID NO: 1 is L70P. In certain embodiments, the amino acid sequence variant of SEQ ID NO: 1 is Q9E. In certain embodiments, the amino acid sequence variant of SEQ ID NO: 1 is Q9A. In certain embodiments, the amino acid sequence variant of SEQ ID NO: 1 is Q9N. In certain embodiments, the amino acid sequence variant of SEQ ID NO: 1 is R15K. In certain embodiments, the amino acid sequence variant of SEQ ID NO: 1 is R15A. In certain embodiments, the amino acid sequence variant of SEQ ID NO: 1 is R15M. In certain embodiments, the amino acid sequence variant of SEQ ID NO: 1 is K18R. In certain embodiments, the amino acid sequence variant of SEQ ID NO: 1 is K18A. In certain embodiments, the amino acid sequence variant of SEQ ID NO: 1 is K18M. In certain embodiments, the amino acid sequence variant of SEQ ID NO: 1 is K23R. In certain embodiments, the amino acid sequence variant of SEQ ID NO: 1 is K23A. In certain embodiments, the amino acid sequence variant of SEQ ID NO: 1 is K23M.

In certain embodiments, said transcription activating domain from a second transcription factor is from VP64, p65, and Rta, and combinations thereof. In certain embodiments, said transcription activating domain comprises the amino acid sequence set forth in: RAGKPIPNPLLGLDSTDALDDFDLDMLGSDALDDFDLDMLGSDALDDFDLDMLGSDAL DDFDLDMLGSPKKKRKVGSQYLPDTDDRHRIEEKRKRTYETFKSIMKKSPFSGPTDPRPPPR RIAVPSRSSASVPKPAPQPYPFTSSLSTINYDEFPTMVFPSGQISQASALAPAPPQVLPQAPAP APAPAMVSALAQAPAPVPVLAPGPPQAVAPPAPKPTQAGEGTLSEALLQLQFDDEDLGALL GNSTDPAVFTDLASVDNSEFQQLLNQGIPVAPHTTEPMLMEYPEAITRLVTGAQRPPDPAPA PLGAPGLPNGLLSGDEDFSSIADMDFSALLSQISSGSGSGSRDSREGMFLPKPEAGSAISDVFE GREVCQPKRIRPFHPPGSPWANRPLPASLAPTPTGPVHEPVGSLTPAPVPQPLDPAPAVTPEA SHLLEDPDEETSQAVKALREMADTVIPQKEEAAICGQMDLSHPPPRGHLDELTTTLESMTED LNLDSPLTPELNEILDTFLNDECLLHAMHISTGLSIFDTSLF, SEQ ID NO: 14.

In certain embodiments, the nucleic acids described herein encode a transcription factor with a VPR amino acid sequence at least 90% 95%, 97%, 98%, 99%, or 100% identical to that set forth in SEQ ID NO: 14. In certain embodiments, the nucleic acids described herein encode a transcription factor with a VPR amino acid sequence at least 90% identical to that set forth in SEQ ID NO: 14. In certain embodiments, the nucleic acids described herein encode a transcription factor with a VPR amino acid sequence at least 95% identical to that set forth in SEQ ID NO: 14. In certain embodiments, the nucleic acids described herein encode a transcription factor with a VPR amino acid sequence at least 97% identical to that set forth in SEQ ID NO: 14. In certain embodiments, the nucleic acids described herein encode a transcription factor with a VPR amino acid sequence at least 98% identical to that set forth in SEQ ID NO: 14. In certain embodiments, the nucleic acids described herein encode a transcription factor with a VPR amino acid sequence at least 99% identical to that set forth in SEQ ID NO: 10. In certain embodiments, the nucleic acids described herein encode a transcription factor with a VPR amino acid sequence 100% identical to that set forth in SEQ ID NO: 14.

In certain embodiments, a transcription activating domain on a synthetic transcription factor comprises an amino acid sequence variant that increases or decreases transcriptional activation. In certain embodiments, said transcription activating domain comprising an amino acid sequence variant that increases or decreases transcriptional activation is a sequence variant of SEQ ID NO: 14.

In certain embodiments, a synthetic transcription factor encoded by a nucleic acid sequence of a transcription factor nucleic acid comprises a polypeptide sequence that destabilizes said synthetic transcription factors, also termed a “degron.” In certain embodiments, said polypeptide sequence that destabilizes said transcription factor comprises a PEST polypeptide sequence. A PEST polypeptide sequence is a polypeptide sequence containing a plurality of amino acids, wherein said polypeptide sequence is rich in the amino acids proline, glutamic acid, serine, and/or threonine. In certain embodiments, said polypeptide sequence that destabilizes said transcription factor comprises a CL1 polypeptide sequence. A CL1 polypeptide sequence may act as a degradation signal, leading to a shorter half-life of the resulting synthetic transcription factor. In certain embodiments, said polypeptide sequence that destabilizes said synthetic transcription factor aids in reduction of background signal of a reporter.

In certain embodiments, said synthetic transcription factor comprises a GAL4-VP16 chimeric transcription factor. In certain embodiments, the transcription factor comprises a GAL4-VPR chimeric transcription factor. The sequence of the Gal4-VPR chimeric transcription factor is given by the sequence set forth in MKLLSSIEQACDICRLKKLKCSKEKPKCAKCLKNNWECRYSPKTKRSPLTRAHLTEVESRLE RLEQLFLLIFPREDLDMILKMDSLQDIKALLTGLFVQDNVNKDAVTDRLASVETDMPLTLRQ HRISATSSSEESSNKGQRQLTVSASGSGRAGKPIPNPLLGLDSTDALDDFDLDMLGSDALDD FDLDMLGSDALDDFDLDMLGSDALDDFDLDMLGSPKKKRKVGSQYLPDTDDRHRIEEKRK RTYETFKSIMKKSPFSGPTDPRPPPRRIAVPSRSSASVPKPAPQPYPFTSSLSTINYDEFPTMVF PSGQISQASALAPAPPQVLPQAPAPAPAPAMVSALAQAPAPVPVLAPGPPQAVAPPAPKPTQ AGEGTLSEALLQLQFDDEDLGALLGNSTDPAVFTDLASVDNSEFQQLLNQGIPVAPHTTEPM LMEYPEAITRLVTGAQRPPDPAPAPLGAPGLPNGLLSGDEDFSSIADMDFSALLSQISSGSGS GSRDSREGMFLPKPEAGSAISDVFEGREVCQPKRIRPFHPPGSPWANRPLPASLAPTPTGPVH EPVGSLTPAPVPQPLDPAPAVTPEASHLLEDPDEETSQAVKALREMADTVIPQKEEAAICGQ MDLSHPPPRGHLDELTTTLESMTEDLNLDSPLTPELNEILDTFLNDECLLHAMHISTGLSIFDT SLF, SEQ ID NO: 10. In certain embodiments, the nucleic acids described herein encode a transcription factor with an amino acid sequence at least 90% 95%, 97%, 98%, 99%, or 100% identical to that set forth in SEQ ID NO: 10. In certain embodiments, the nucleic acids described herein encode a transcription factor with an amino acid sequence at least 90% identical to that set forth in SEQ ID NO: 10. In certain embodiments, the nucleic acids described herein encode a transcription factor with an amino acid sequence at least 95% identical to that set forth in SEQ ID NO: 10. In certain embodiments, the nucleic acids described herein encode a transcription factor with an amino acid sequence at least 97% identical to that set forth in SEQ ID NO: 10. In certain embodiments, the nucleic acids described herein encode a transcription factor with an amino acid sequence at least 98% identical to that set forth in SEQ ID NO: 10. In certain embodiments, the nucleic acids described herein encode a transcription factor with an amino acid sequence at least 99% identical to that set forth in SEQ ID NO: 10. In certain embodiments, the nucleic acids described herein encode a transcription factor with an amino acid sequence 100% identical to that set forth in SEQ ID NO: 10.

In certain embodiments, said synthetic transcription factor comprises a Gal4 DNA binding domain given by the amino acid sequence set forth in SEQ ID NO: 1. In certain embodiments, said synthetic transcription factor comprises a DNA binding domain with an amino acid sequence at least 90% 95%, 97%, 98%, 99%, or 100% identical to that set forth in SEQ ID NO: 1. In certain embodiments, said synthetic transcription factor comprises a DNA binding domain with an amino acid sequence at least 90% identical to that set forth in SEQ ID NO: 1. In certain embodiments, said synthetic transcription factor comprises a DNA binding domain with an amino acid sequence at least 95% identical to that set forth in SEQ ID NO: 1. In certain embodiments, said synthetic transcription factor comprises a DNA binding domain with an amino acid sequence at least 97% identical to that set forth in SEQ ID NO: 1. In certain embodiments, said synthetic transcription factor comprises a DNA binding domain with an amino acid sequence at least 98% identical to that set forth in SEQ ID NO: 1. In certain embodiments, said synthetic transcription factor comprises a DNA binding domain with an amino acid sequence at least 99% identical to that set forth in SEQ ID NO: 1. In certain embodiments, said synthetic transcription factor comprises a DNA binding domain with an amino acid sequence 100% identical to that set forth in SEQ ID NO: 1.

In certain embodiments, said synthetic transcription factor comprises a transcription activating domain from VP64 given by the amino acid sequence set forth in RAGKPIPNPLLGLDSTDALDDFDLDMLGSDALDDFDLDMLGSDALDDFDLDMLGSDALDD FDLDMLGSPKKKRKV, SEQ ID NO: 11. In certain embodiments, said synthetic transcription factor comprises a transcription activating domain with an amino acid sequence at least 90% 95%, 97%, 98%, 99%, or 100% identical to that set forth in SEQ ID NO: 11. In certain embodiments, said synthetic transcription factor comprises a transcription activating domain with an amino acid sequence at least 90% identical to that set forth in SEQ ID NO: 11. In certain embodiments, said synthetic transcription factor comprises a transcription activating domain with an amino acid sequence at least 95% identical to that set forth in SEQ ID NO: 11. In certain embodiments, said synthetic transcription factor comprises a transcription activating domain with an amino acid sequence at least 97% identical to that set forth in SEQ ID NO: 11. In certain embodiments, said synthetic transcription factor comprises a transcription activating domain with an amino acid sequence at least 98% identical to that set forth in SEQ ID NO: 11. In certain embodiments, said synthetic transcription factor comprises a transcription activating domain with an amino acid sequence at least 99% identical to that set forth in SEQ ID NO: 11. In certain embodiments, said synthetic transcription factor comprises a transcription activating domain with an amino acid sequence 100% identical to that set forth in SEQ ID NO: 11.

In certain embodiments, said synthetic transcription factor comprises a transcription activating domain from p65 given by the amino acid sequence set forth in QYLPDTDDRHRIEEKRKRTYETFKSIMKKSPFSGPTDPRPPPRRIAVPSRSSASVPKPAPQPYP FTSSLSTINYDEFPTMVFPSGQISQASALAPAPPQVLPQAPAPAPAPAMVSALAQAPAPVPVL APGPPQAVAPPAPKPTQAGEGTLSEALLQLQFDDEDLGALLGNSTDPAVFTDLASVDNSEF QQLLNQGIPVAPHTTEPMLMEYPEAITRLVTGAQRPPDPAPAPLGAPGLPNGLLSGDEDFSSI ADMDFSALLSQISS, SEQ ID NO: 12. In certain embodiments, said synthetic transcription factor comprises a transcription activating domain with an amino acid sequence at least 90% 95%, 97%, 98%, 99%, or 100% identical to that set forth in SEQ ID NO: 12. In certain embodiments, said synthetic transcription factor comprises a transcription activating domain with an amino acid sequence at least 90% identical to that set forth in SEQ ID NO: 12. In certain embodiments, said synthetic transcription factor comprises a transcription activating domain with an amino acid sequence at least 95% identical to that set forth in SEQ ID NO: 12. In certain embodiments, said synthetic transcription factor comprises a transcription activating domain with an amino acid sequence at least 97% identical to that set forth in SEQ ID NO: 12. In certain embodiments, said synthetic transcription factor comprises a transcription activating domain with an amino acid sequence at least 98% identical to that set forth in SEQ ID NO: 12. In certain embodiments, said synthetic transcription factor comprises a transcription activating domain with an amino acid sequence at least 99% identical to that set forth in SEQ ID NO: 12. In certain embodiments, said synthetic transcription factor comprises a transcription activating domain with an amino acid sequence 100% identical to that set forth in SEQ ID NO: 12.

In certain embodiments, said synthetic transcription factor comprises a transcription activating domain from Rta given by the amino acid sequence set forth in RDSREGMFLPKPEAGSAISDVFEGREVCQPKRIRPFHPPGSPWANRPLPASLAPTPTGPVHEP VGSLTPAPVPQPLDPAPAVTPEASHLLEDPDEETSQAVKALREMADTVIPQKEEAAICGQMD LSHPPPRGHLDELTTTLESMTEDLNLDSPLTPELNEILDTFLNDECLLHAMHISTGLSIFDTSL F, SEQ ID NO: 13. In certain embodiments, said synthetic transcription factor comprises a transcription activating domain with an amino acid sequence at least 90% 95%, 97%, 98%, 99%, or 100% identical to that set forth in SEQ ID NO: 13. In certain embodiments, said synthetic transcription factor comprises a transcription activating domain with an amino acid sequence at least 90% identical to that set forth in SEQ ID NO: 13. In certain embodiments, said synthetic transcription factor comprises a transcription activating domain with an amino acid sequence at least 95% identical to that set forth in SEQ ID NO: 13. In certain embodiments, said synthetic transcription factor comprises a transcription activating domain with an amino acid sequence at least 97% identical to that set forth in SEQ ID NO: 13. In certain embodiments, said synthetic transcription factor comprises a transcription activating domain with an amino acid sequence at least 98% identical to that set forth in SEQ ID NO: 13. In certain embodiments, said synthetic transcription factor comprises a transcription activating domain with an amino acid sequence at least 99% identical to that set forth in SEQ ID NO: 13. In certain embodiments, said synthetic transcription factor comprises a transcription activating domain with an amino acid sequence 100% identical to that set forth in SEQ ID NO: 13.

### Synthetic Transcription Factor Promoter Nucleotide Sequences

A synthetic transcription factor promoter nucleotide sequence is a sequence of nucleic acids capable of being bound by a synthetic transcription factor. In certain embodiments, said synthetic transcription factor nucleotide sequence is not bound by endogenous transcription factors. Said synthetic transcription factor promoter nucleotide sequence aids in recruitment of said synthetic transcription factor in order to activate transcription of a reporter molecule. Said reporter molecule is encoded on a nucleic acid positioned 3′ of said synthetic transcription factor promoter nucleotide sequence.

In the methods, nucleic acids, and systems described herein, a synthetic transcription factor promoter nucleotide sequence is encoded on a reporter nucleic acid. Said synthetic transcription factor promoter nucleotide sequence is able to be bound by a synthetic transcription factor encoded on a transcription factor nucleic acid. Said synthetic transcription factor promoter nucleotide sequence is positioned 5′ of a nucleotide sequence encoding a reporter. In certain embodiments, said synthetic transcription factor promoter nucleotide sequence is not bound by endogenous transcription factors. In certain embodiments, said synthetic transcription factor is highly specific for said synthetic transcription factor promoter nucleotide sequence.

In certain embodiments, said synthetic transcription factor promoter nucleotide sequence is able to be bound by Gal4, PPR1, Lac9, or LexA. In certain embodiments, said synthetic transcription factor is able to be bound by a polypeptide comprising the amino acid sequence set forth in SEQ ID NO: 1.

In certain embodiments, said synthetic transcription factor promoter nucleotide sequence is able to be bound by an amino acid sequence variant of Gal4, PPR1, Lac9, or LexA. In certain embodiments, said synthetic transcription factor promoter nucleotide sequence is able to be bound an amino acid sequence variant of SEQ ID NO: 1.

### Reporter Elements

The reporter nucleic acid minimally comprises a regulatory element that is able to be bound by a synthetic transcription factor and a nucleotide sequence encoding a reporter. Said nucleotide sequence encoding a reporter is downstream of said regulatory element that is able to be bound by said synthetic transcription factor. Said synthetic transcription factor regulates expression of said reporter.

In certain embodiments, the nucleotide sequence encoding a reporter comprises a reporter gene. In certain embodiments, said reporter gene encodes a reporter selected from a fluorescent protein, a luciferase protein, a beta-galactosidase, a beta-glucuronidase, a chloramphenicol acetyltransferase, and a secreted placental alkaline phosphatase. These reporter proteins can be assayed for a specific enzymatic activity or in the case of a fluorescent reporter can be assayed for fluorescent emissions. In certain embodiments, the fluorescent protein comprises a green fluorescent protein (GFP), a red fluorescent protein (RFP), a yellow fluorescent protein (YFP), or a cyan fluorescent protein (CFP).

In certain embodiments, the nucleotide sequence encoding a reporter gene comprises a nucleotide sequence encoding a unique sequence identifier (UMI). In certain embodiments, said UMI is unique to a test polypeptide, wherein said test polypeptide is encoded by said reporter nucleic acid. Generally, said UMI will be between 8 and 20 nucleotides in length, however it may be longer. In certain embodiments, said UMI is 8, 9, 10, 11, 12, 13, 14, 15, 16, 17, 18, 19, 20, or more nucleotides in length. In certain embodiments, said UMI is 8 nucleotides in length. In certain embodiments, said UMI is 9 nucleotides in length. In certain embodiments, said UMI is 10 nucleotides in length. In certain embodiments, said UMI is 11 nucleotides in length. In certain embodiments, said UMI is 12 nucleotides in length. In certain embodiments, said UMI is 13 nucleotides in length. In certain embodiments, said UMI is 14 nucleotides in length. In certain embodiments, said UMI is 15 nucleotides in length. In certain embodiments, said UMI is 16 nucleotides in length. In certain embodiments, said UMI is 17 nucleotides in length. In certain embodiments, said UMI is 18 nucleotides in length. In certain embodiments, said UMI is 19 nucleotides in length. In certain embodiments, said UMI is 20 nucleotides in length. In certain embodiments, said UMI is more than 20 nucleotides in length.

The system described herein can utilize many different regulatory sequences that control activation of the reporter gene through synthetic transcription factor binding. The regulatory sequence is one that can be bound by the synthetic transcription factor polypeptide. Generally, it will be configured so that the regulatory sequence is 5′ to the UMI, the reporter gene, or both. In certain embodiments, the regulatory sequence comprises a Gal4-, PPR1-, or LexA-UAS, which is able to be bound by a synthetic transcription factor.

In certain embodiments, the reporter comprises a fluorescent protein, a luciferase protein, a beta-galactosidase, a beta-glucuronidase, a chloramphenicol acetyltransferase, or a secreted placental alkaline phosphatase, and a UMI. In certain embodiments, said UMI is encoded on the reporter nucleic acid 5′ of the fluorescent protein, luciferase protein, beta-galactosidase, beta-glucuronidase, chloramphenicol acetyl transferase, or secreted placental alkaline phosphatase. In certain embodiments, a nucleotide sequence encoding the fluorescent protein, luciferase protein, beta-galactosidase, beta-glucuronidase, chlorampheniol acetyltransferase, or secreted placental alkaline phosphatase is 5′ of said UMI.

A UMI allows for multiplexing of different transcriptional relay systems within the same assay since transcription of the UMI will indicate association of a specific relay system with the reporter. The UMI can be any length that allows for sufficient diversity to allow multiplexed determination of different transcriptional relay systems within the same assay. Said length should be sufficient to differentiate between at least 100, 500, 1,000, 2,000, 3,000, 4,000, 5,000, 6,000, 7,000, 8,000, 9,000, or 10,000 transcriptional relay targets. In certain embodiments, said different transcriptional relay systems will be present in different cells. In certain embodiments, said different transcriptional relay systems will be present in the same cell.

Reporter elements may further comprise a 5′ UTR, a 3′UTR or both. The UTR may be heterologous to the reporter element.

### Reporter Activation

Activation of a reporter molecule can be determined using standard assays to detect a luciferase protein, a beta-galactosidase protein, a beta-glucuronidase protein, a chloramphenicol acetyltransferase protein, a secreted placental alkaline phosphatase protein. Generally, these are enzymatic assays where a detectable signal is produced based upon the proteins enzymatic activity towards a substrate. For example, luciferase expression can be measured in the presence of a luciferase substrate by a luminometer. A fluorescent reporter does not require a substrate, and the signal can be measured by fluorescence microscopy or a fluorescent plate reader. Fluorescent reporters are particularly useful for measuring reporter activation in live cells.

In embodiments wherein a reporter molecule comprises a unique RNA sequence, reporter activation can be measured in any suitable way that allows sequence determination of the unique RNA sequence, with a preference for methods that allow sequence determination in a multiplex fashion. Such methods include high throughput sequencing methods that can generate information on at least about 100,000, 1,000,000, 10,000,000, or 100,000,000 DNA or RNA bases in a 24-hour period. In certain embodiments, a next-generation sequencing technology is used to determine the sequence of the unique RNA sequence. Next generation sequencing encompasses many kinds of sequencing such as pyrosequencing, sequencing-by-synthesis, single-molecule sequencing, second-generation sequencing, nanopore sequencing, sequencing by ligation, or sequencing by hybridization. Next-generation sequencing platforms include those commercially available from Illumina (RNA-Seq) and Helicos (Digital Gene Expression or “DGE”). Next generation sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos Biosciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina, Inc. as described in U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. Such methods and apparatuses are provided here by way of example and are not intended to be limiting.

### Markers

In certain embodiments, the nucleic acids described herein additionally comprise one or more additional genes that encode a selecting polypeptide or a marking polypeptide. In certain embodiments, the nucleic acids described herein additionally comprise one or more additional genes that encode a polypeptide that confers antibiotic resistance to a transfected cell. For example, the nucleic acids can comprise a selectable marker such as an antibiotic resistance gene that confers antibiotic resistance to neomycin/G418 resistance, puromycin resistance, zeocin resistance, or blasticidin resistance. In certain embodiments, the nucleic acids described herein additionally comprise one or more additional genes that encode a polypeptide that comprises an epitope tag that is expressed on the cell surface. This allows for affinity purification or cell sorting to collect cells that have been transfected with the nucleic acids described. In certain embodiments, the epitope tag comprises a c-Myc tag, a Hemagglutinin (HA) tag, a histidine tag, a V5 tag, or a FLAG tag. In certain embodiments, the nucleic acids described herein additionally comprise one or more additional promotorless genes that encode a fluorescent polypeptide. Such genes are useful when transfection is intended to lead to integration and is targeted for a specific location or landing pad. In these cases the “landing pad” in the cells genome comprises a promoter that can complement the lack of promotor in the pomotorless gene, and lead to expression of the promotorless gene only when integrated into the intended genomic location. Cells with correct integration can be selected by flow cytometry and cell sorting. This type of marker can also ensure that only a single copy of an intended nucleic acid is integrated in the genome, and help avoid ectopic overexpression. In certain embodiments, a nucleic acid encoding a bait polypeptide comprises: a gene that encodes a polypeptide that confers antibiotic resistance to a transfected cell; a gene that encodes a polypeptide that comprises an epitope tag that is expressed on the cell surface; or a promotorless gene that encodes a fluorescent polypeptide.

### Cells

Cells useful in the method described herein are generally those that are able to be easily rendered transgenic with one or more exogenous nucleic acids encoding a synthetic transcription factor and a reporter element. The system nucleic acid(s) encoding a synthetic transcription factor and a reporter element can be transfected or transduced into suitable cell line using methods known in the art, such as calcium phosphate transfection, lipid based transfection (e.g., Lipofectamine™, Lipofectamine-2000™, Lipofectamine-3000™, or Fugene® HD), electroporation, or viral transduction. The cell can also be a population of cells of the same type grown to confluency or near confluency in an appropriate tissue culture vessel.

In certain embodiments, the cell used comprises a stable integration of either the nucleic acid encoding the synthetic transcription factor, the nucleic acid comprising the reporter element, or both. Stable cell lines can be made using random integration of a linearized plasmid, virally or transposon directed integration, or directed integration, for example using site specific recombination between an AttP and an AttB site. In certain embodiments, either of the nucleic acids are encoded at a safe landing site such as the AAVS1 site.

In certain embodiments, the cell or cell population used in the system is a eukaryotic cell. In certain embodiments, the cell or cell population is a mammalian cell. In certain embodiments, the cell or cell population is a human cell. In certain embodiments, the cell or cell population is SH-SY5Y, Human neuroblastoma; Hep G2, Human Caucasian hepatocyte carcinoma; 293 (also known as HEK 293), Human Embryo Kidney; RAW 264.7, Mouse monocyte macrophage; HeLa, Human cervix epitheloid carcinoma; MRC-5 (PD 19), Human fetal lung; A2780, Human ovarian carcinoma; CACO-2, Human Caucasian colon adenocarcinoma; THP 1, Human monocytic leukemia; A549, Human Caucasian lung carcinoma; MRC-5 (PD 30), Human fetal lung; MCF7, Human Caucasian breast adenocarcinoma; SNL 76/7, Mouse SIM strain embryonic fibroblast; C2C12, Mouse C3H muscle myoblast; Jurkat E6.1, Human leukemic T cell lymphoblast; U937, Human Caucasian histiocytic lymphoma; L929, Mouse C3H/An connective tissue; 3T3 L1, Mouse Embryo; HL60, Human Caucasian promyelocytic leukaemia; PC-12, Rat adrenal phaeochromocytoma; HT29, Human Caucasian colon adenocarcinoma; OE33, Human Caucasian oesophageal carcinoma; OE19, Human Caucasian oesophageal carcinoma; NIH 3T3, Mouse Swiss NIH embryo; MDA-MB-231, Human Caucasian breast adenocarcinoma; K562, Human Caucasian chronic myelogenous leukemia; U-87 MG, Human glioblastoma astrocytoma; MRC-5 (PD 25), Human fetal lung; A2780cis, Human ovarian carcinoma; B9, Mouse B cell hybridoma; CHO-K1, Hamster Chinese ovary; MDCK, Canine Cocker Spaniel kidney; 1321N1, Human brain astrocytoma; A431, Human squamous carcinoma; ATDC5, Mouse 129 teratocarcinoma AT805 derived; RCC4 PLUS VECTOR ALONE, Renal cell carcinoma cell line RCC4 stably transfected with an empty expression vector, pcDNA3, conferring neomycin resistance; HUVEC (5200-05n), Human Pre-screened Umbilical Vein Endothelial Cells (HUVEC); neonatal; Vero, Monkey African Green kidney; RCC4 PLUS VHL, Renal cell carcinoma cell line RCC4 stably transfected with pcDNA3-VHL; Fao, Rat hepatoma; J774A.1, Mouse BALB/c monocyte macrophage; MC3T3-E1, Mouse C57BL/6 calvaria; J774.2, Mouse BALB/c monocyte macrophage; PNT1A, Human post pubertal prostate normal, immortalised with SV40; U-2 OS, Human Osteosarcoma; HCT 116, Human colon carcinoma; MA104, Monkey African Green kidney; BEAS-2B, Human bronchial epithelium, normal; NB2-11, Rat lymphoma; BHK 21 (clone 13), Hamster Syrian kidney; NS0, Mouse myeloma; Neuro 2a, Mouse Albino neuroblastoma; SP2/0-Ag14, Mouse×Mouse myeloma, non-producing; T47D, Human breast tumor; 1301, Human T-cell leukemia; MDCK-II, Canine Cocker Spaniel Kidney; PNT2, Human prostate normal, immortalized with SV40; PC-3, Human Caucasian prostate adenocarcinoma; TF1, Human erythroleukaemia; COS-7, Monkey African green kidney, SV40 transformed; MDCK, Canine Cocker Spaniel kidney; HUVEC (200-05n), Human Umbilical Vein Endothelial Cells (HUVEC); neonatal; NCI-H322, Human Caucasian bronchioalveolar carcinoma; SK.N. SH, Human Caucasian neuroblastoma; LNCaP.FGC, Human Caucasian prostate carcinoma; 0E21, Human Caucasian oesophageal squamous cell carcinoma; PSN1, Human pancreatic adenocarcinoma; ISHIKAWA, Human Asian endometrial adenocarcinoma; MFE-280, Human Caucasian endometrial adenocarcinoma; MG-63, Human osteosarcoma; RK 13, Rabbit kidney, BVDV negative; EoL-1 cell, Human eosinophilic leukemia; VCaP, Human Prostate Cancer Metastasis; tsA201, Human embryonal kidney, SV40 transformed; CHO, Hamster Chinese ovary; HT 1080, Human fibrosarcoma; PANC-1, Human Caucasian pancreas; Saos-2, Human primary osteogenic sarcoma; Fibroblast Growth Medium (116K-500), Fibroblast Growth Medium Kit; ND7/23, Mouse neuroblastoma×Rat neuron hybrid; SK-OV-3, Human Caucasian ovary adenocarcinoma; COV434, Human ovarian granulosa tumor; Hep 3B, Human hepatocyte carcinoma; Vero (WHO), Monkey African Green kidney; Nthy-ori 3-1, Human thyroid follicular epithelial; U373 MG (Uppsala), Human glioblastoma astrocytoma; A375, Human malignant melanoma; AGS, Human Caucasian gastric adenocarcinoma; CAKI 2, Human Caucasian kidney carcinoma; COLO 205, Human Caucasian colon adenocarcinoma; COR-L23, Human Caucasian lung large cell carcinoma; IMR 32, Human Caucasian neuroblastoma; QT 35, Quail Japanese fibrosarcoma; WI 38, Human Caucasian fetal lung; HMVII, Human vaginal malignant melanoma; HT55, Human colon carcinoma; TK6, Human lymphoblast, thymidine kinase heterozygote; SP2/0-AG14 (AC-FREE), Mouse×mouse hybridoma non-secreting, serum-free, animal component (AC) free; AR42J, or Rat exocrine pancreatic tumor, or any combination thereof.

Described herein are cells and cell lines comprising a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor. In certain embodiments, the cell line is a mammalian cell line. In certain embodiments, the response element regulated promoter is a cAMP response element nucleotide sequence, an NFAT transcription factor response element nucleotide sequence, a FOS promoter nucleotide sequence, or a serum response element nucleotide sequence. In certain embodiments, the response element regulated promoter is an NFAT response element regulated promoter. In certain embodiments, the cell line comprises a reporter nucleic acid comprising a synthetic transcription factor promoter nucleotide sequence and a nucleotide sequence encoding a reporter, wherein said synthetic transcription factor promoter nucleotide sequence is 5′ to said nucleotide sequence encoding said reporter, and wherein said synthetic transcription factor promoter nucleotide sequence is able to be bound by said synthetic transcription factor.

In certain embodiments, the cell line comprises a high basal reporter activity. In certain embodiments, the high basal reporter activity is at least about 5%, 10%, 20%, 25%, 30%, 40%, 50%, 60%, 70%, 80%, 90%, 100%, 200%, 300%, 400%, 500% greater than background, wherein background is the level of reporter activity observed for a cell or cell line that does not comprise the reporter. For such comparisons, generally the cell or cell line used as a comparator will be parental to the cell line comprising the reporter (e.g., HEK293 with reporter vs. HEK293 without reporter).

In certain embodiments, the cell line comprises a high basal reporter activity. In certain embodiments, the high basal reporter activity is at least about 2×, 3×, 4×, 5×, 6×, 7×, 8×, 9×, 10×, 15×, 20×, 25×, 30×, 32×, 50×, 75×, 100×, 200×, 500×, 750×, 1,000×, 2,000×, 5,000×10,000×, or 20,000× greater than background, wherein background is the level of reporter activity observed for a cell or cell line that does not comprise the reporter. In certain embodiments, the cell line comprises a high basal reporter activity. In certain embodiments, the high basal reporter activity is at least about 30× greater than background, wherein background is the level of reporter activity observed for a cell or cell line that does not comprise the reporter. In certain embodiments, the high basal reporter activity is at least about 32× greater than background, wherein background is the level of reporter activity observed for a cell or cell line that does not comprise the reporter. For such comparisons, generally the cell or cell line used as a comparator will be parental to the cell line comprising the reporter (e.g., HEK293 with reporter vs. HEK293 without reporter).

In certain embodiments, the cell line comprises low variance in basal reporter activity. In certain embodiments, the low variance in basal reporter activity is a biological coefficient of variance less than about 0.6. In certain embodiments, the low variance in basal reporter activity is a biological coefficient of variance less than about 0.5. In certain embodiments, the low variance in basal reporter activity is a biological coefficient of variance less than about 0.4. In certain embodiments, the low variance in basal reporter activity is a biological coefficient of variance less than about 0.3. In certain embodiments, the low variance in basal reporter activity is a biological coefficient of variance less than about 0.2. In certain embodiments, the low variance in basal reporter activity is a biological coefficient of variance less than about 0.1.

Without being bound by theory reductions in variance and high levels of basal activity can be gained by selecting clonal cell lines that comprise at least 2, 3, 4, 5, or more copies of comprising a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor. In certain embodiments, the response element regulated promoter is a cAMP response element nucleotide sequence, a NFAT transcription factor response element nucleotide sequence, a FOS promoter nucleotide sequence, or a serum response element nucleotide sequence. In certain embodiments, the response element regulated promoter is an NFAT response element regulated promoter. In certain embodiments, the cell line comprises only 1 copy of a reporter nucleic acid comprising a synthetic transcription factor promoter nucleotide sequence and a nucleotide sequence encoding a reporter. In certain embodiments, the cell line comprises only 2 copies of a reporter nucleic acid comprising a synthetic transcription factor promoter nucleotide sequence and a nucleotide sequence encoding a reporter. In certain embodiments, the cell line comprises a reporter nucleic acid comprising a synthetic transcription factor promoter nucleotide sequence and a nucleotide sequence encoding a reporter maintained in an unintegrated or episomal state. In certain embodiments, the cell line further comprises a nucleic acid encoding the cDNA or otherwise intronless version of cell signaling protein. In certain embodiments, the cell signaling protein is a GPCR or a GPCR subunit.

In certain embodiments, the cell comprises a nucleic acid encoding a G protein coupled receptor family member. G protein-coupled receptors (GPCRs), also known as seven-(pass)-transmembrane domain receptors, are ligand binding cell surface signaling proteins. When a ligand binds to the GPCR it causes a conformational change in the GPCR, which allows it to act as a guanine nucleotide exchange factor (GEF). The GPCR can then activate an associated G protein by exchanging the GDP bound to the G protein for a GTP. The G protein's a subunit, together with the bound GTP, can then dissociate from the β and γ subunits to further affect intracellular signaling proteins or target functional proteins directly depending on the α subunit type (Gαs, Gαi/o, Gαq/11, Gα12/13). There are at least about 800 GPCRs encoded in the human genome, broadly divided into Classes A, B, and C which can be utilized with the systems herein. In certain embodiments, the nucleic acid encoding a G protein coupled receptor family member can be integrated into the genome. In certain embodiments, the nucleic acid encoding a G protein coupled receptor family member can be maintained epsiomally.

In certain embodiments, the cell comprises a nucleic acid encoding a receptor tyrosine kinase family member. Receptor tyrosine kinases (RTKs) are high-affinity cell surface receptors for many polypeptide growth factors, cytokines, and hormones. Receptor tyrosine kinases have been shown not only to be key regulators of normal cellular processes but also to have a critical role in the development and progression of many types of cancer. There are many classes of RTKs any member of which can be utilized in the systems described herein. In certain embodiments, the RTK comprises an RTK class I (EGF receptor family) (ErbB family); RTK class II (Insulin receptor family); RTK class III (PDGF receptor family); RTK class IV (VEGF receptors family); RTK class V (FGF receptor family); RTK class VI (CCK receptor family); RTK class VII (NGF receptor family); RTK class VIII (HGF receptor family); RTK class IX (Eph receptor family); RTK class X (AXL receptor family); RTK class XI (TIE receptor family); RTK class XII (RYK receptor family); RTK class XIII (DDR receptor family); RTK class XIV (RET receptor family); RTK class XV (ROS receptor family); RTK class XVI (LTK receptor family); RTK class XVII (ROR receptor family); RTK class XVIII (MuSK receptor family); RTK class XIX (LMR receptor); or RTK class XX (Undetermined) member. In certain embodiments, the nucleic acid encoding an RTK family member can be integrated into the genome. In certain embodiments, the nucleic acid encoding the RTK family member can be maintained epsiomally.

Also described herein is a mammalian cell line comprising an NFAT response element. In certain embodiments, the mammalian cell line comprising the NFAT response element comprises cb29.

Also described herein is a mammalian cell line comprising an NFAT response element. In certain embodiments, the mammalian cell line comprising the NFAT response element comprises cb37.

### Methods of Using the System

The polynucleotide sequences of the present invention may be utilized when transfected into cells. Transfection can be accomplished by a variety of transfection agents, including without limitation lipofectin, calcium phosphate precipitation, viral transduction, or electroporation. Transfection can be transient or stable. In embodiments where transfection is stable, stablely transfected cells can be frozen or banked for later use.

In certain embodiments, a single nucleic acid relay system is transfected into a population of cells. In certain embodiments, 1, 2, 3, 4, 5, 10, 100, or more nucleic acid relay systems are transfected into a population of cells. In certain embodiments, 2 nucleic acid relay systems are transfected into a population of cells. In certain embodiments, 3 nucleic acid relay systems are transfected into a population of cells. In certain embodiments, 4 nucleic acid relay systems are transfected into a population of cells. In certain embodiments, 5 nucleic acid relay systems are transfected into a population of cells. In certain embodiments where a population of cells is transfected with a plurality of nucleic acid relay systems, said plurality of nucleic acid relay systems comprise different response element regulated promotors. In certain embodiments where said plurality of nucleic acid relay systems comprise different response element regulated promoters, said plurality of nucleic acid relay systems comprise different reporters. In certain embodiments, said different reporters comprise a UMI.

Cell populations transfected with nucleic acids of the present invention can be any size. In certain embodiments, cell populations comprise 1,000, 10,000, 100,000, 1,000,000, 10,000,000 or more cells. In certain embodiments, at least about 1,000 or more cells are transfected with one or more transcriptional relay systems. In certain embodiments, at least about 10,000 or more cells are transfected with one or more transcriptional relay systems. In certain embodiments, at least about 100,000 or more cells are transfected with one or more transcriptional relay systems. In certain embodiments, at least about 1,000,000 or more cells are transfected with one or more transcriptional relay systems. In certain embodiments, at least about 10,000,000 or more cells are transfected with one or more transcriptional relay systems.

In certain embodiments, the nucleic acid systems of the present invention can be utilized in multiwell plate experiments. Non-limiting examples of multiwell plates compatible with the nucleic acid relay systems of the present invention include 6, 12, 24, 48, 96, 384, or 1,536 well plates. In certain embodiments, each well of a multiwell plate comprises a cell population transfected with a single transcriptional relay system. In certain embodiments, each well of a multiwell plate comprises a cell population transfected with a plurality of transcriptional relay systems. In certain embodiments, each well comprises multiple cell populations, each cell population transfected with a single nucleic acid relay system. In certain embodiments, each well comprises multiple cell populations, each cell population transfected with a plurality of nucleic acid relay systems.

In certain embodiments, test agents are applied to cells transfected with transcriptional relay systems of the present invention. In certain embodiments, level of activation of transcription of a reporter molecule is measured after said cells are contacted by said test agent. In certain embodiments, said test agent is a chemical, small-molecule, biological molecule, polypeptide, polynucleotide, aptamer, or any combination thereof. In certain embodiments, a single test agent is applied to a population of cells. In certain embodiments, a plurality of test agents are applied to a population of cells.

In certain embodiments, the transcriptional relay system of the present invention is adapted for measuring responses of GPCRs to test agents. The nucleic acid systems of the present invention can be adapted for use with any GPCR receptor. In certain embodiments, said transcriptional relay systems are adapted for use with GPCR receptors by utilizing a cAMP response element regulated promoter. Non-limiting examples of GPCRs include 5-hydroxytryptamine receptors, acetylcholine receptors, adenosine receptors, adrenoceptors, angiotensin receptors, apelin receptor, bile acid receptor, bombesin receptors, bradykinin receptors, cannabinoid receptors, chemerin receptors, chemokine receptors, cholecystokinin receptors, dopamine receptors, endothelin receptors, formylpeptide receptors, free fatty acid receptors, galanin receptors, ghrelin receptor, glycoprotein hormone receptors, gonadotrophin-releasing hormone receptors, GPR18, GPR55, GPR119, G protein-coupled estrogen receptor, histamine receptors, hydroxycarboxylic acid receptors, kisspeptin receptors, leukotriene receptors, LPA receptors, S1P receptors, melanin-concentrating hormone receptors, melanocortin receptors, melatonin receptors, motilin receptor, neuromedin U receptors, neuropeptide FF/neuropeptide AF receptors, neuropeptide S receptor, neuropeptide W/neuropeptide B receptors, neuropeptide Y receptors, neurotensin receptors, opioid receptors, opsin receptors, orexin receptors, oxoglutarate receptor, P2Y receptors, platelet-activating factor receptor, prokineticin receptors, prolactin-releasing peptide receptor, prostanoid receptors, proteinase-activated receptors, QRFP receptor, relaxin family peptide receptors, somatostatin receptors, succinate receptors, tachykinin receptors, thyrotropin-releasing hormone receptors, trace amine receptors, urotensin receptor, vasopressin and oxytocin receptors, calcitonin receptors, corticotropin-releasing factor receptors, glucagon receptor family, parathyroid hormone receptors, VIP and PACAP receptors, calcium-sensing receptors, GABA_{B} receptors, metabotropic glutamate receptors, taste 1 receptors, frizzled class receptors, adhesion class GPCRs, orphan receptors, and any combination thereof.

The nucleic acids of the present invention are compatible with many vectors common in the art. Non-limiting examples of vectors include genomic integrated vectors, episomal vectors, plasmids, viral vectors, cosmids, bacterial artificial chromosomes, and yeast artificial chromosomes. Non-limiting examples of viral vectors compatible with the nucleic acids of the present invention include vectors derived from lentiviruses, retroviruses, adenoviruses, and adeno-associated viruses. In certain embodiments, the nucleic acids of the present invention are present on vectors comprising sequences that direct site specific integration into a defined location or a restricted set of sites in the genome (e.g. AttP-AttB recombination).

In certain embodiments, a transcriptional relay system as described herein is incorporated into a single vector. In certain embodiments, said single vector is transfected into a cell transiently. In certain embodiments, said single vector is transfected into a cell stably.

In certain embodiments, said transcriptional relay system is divided across two vectors. In certain embodiments, a transcription factor nucleic acid comprising a response element regulated promoter nucleotide sequence and a nucleotide sequence encoding a synthetic transcription factor, is incorporated into a first vector, and a reporter nucleic acid comprising a synthetic transcription factor promoter nucleotide sequence and a nucleotide sequence encoding a reporter in incorporated into a second vector. In certain embodiments, said first vector and said second vector are transiently transfected into a cell. In certain embodiments, said first vector and said second vector are stably transfected into a cell. In certain embodiments, said first vector is transfected into a cell stably and said second vector is transfected into a cell transiently. In certain embodiments, said first vector is transfected into a cell transiently and said second vector is transfected into a cell stably.

Vectors comprising the transcriptional relay systems described herein or portions thereof may be constructed using many well-known molecular biology techniques. Detailed protocols for numerous such procedures, including amplification, cloning, mutagenesis, transformation, and the like, are described in, e.g., in Ausubel et al. Current Protocols in Molecular Biology (supplemented through 2012) John Wiley & Sons, New York 10 (“Ausubel”); Sambrook et al. Molecular Cloning —A Laboratory Manual (4th Ed.), Vol. 1-3, Cold Spring Harbor Laboratory, Cold Spring Harbor, N.Y., 2012 (“Sambrook”); and Abelson et al. Guide to Molecular Cloning Techniques (Methods in Enzymology) volume 152 Academic Press, Inc., San Diego, Calif. (“Abelson”).

### EXAMPLES

The following illustrative examples are representative of embodiments of compositions and methods described herein and are not meant to be limiting in any way.

### Example 1—Example GPCR Receptor Screen for CRE Activation

In this example, a transcriptional relay system comprising a nucleic acid, as configured in FIGS. 1A and 1B, is used to screen for potential compounds that induce GPCR signaling. For this example, the nucleic acid of FIG. 1A comprises a cAMP response element (CRE) activation that results in expression of a synthetic transcription factor Gal4-VPR (comprising Gal4 DNA binding domain and the chimeric activation domain VP64-p65-Rta). The nucleic acid of FIG. 1B comprises a promoter able to be bound and activated by the Gal4-VPR synthetic transcription factor, which results in expression of a reporter element that comprises a luciferase gene and a gene encoding a UMI. The cells used comprise a stably integrated nucleic acid(s) that encodes the system of FIGS. 1A and 1B, and a given GPCR. Each UMI is associated with a given GPCR allowing for CRE expression to be mapped to a particular GPCR. This allows for multiplexing of the assay.

On day 1, plate cells in a 96-well assay plate at 35,000 cells/well in DMEM. On day 2, exchange the media to 0.5% FBS+DMEM. On day 3, remove the media and add a test compound at a desired concentration in 25 uL of Opti-mem. After about 4 hours, remove the media and replace with lysis buffer for RNA extraction. RNA is extracted using standard methods or kits, and subsequently quantified by a standard assay. RNAseq is then performed on an Illumina MiSeq after sequencing library preparation.

### Example 2—Example GPCR Receptor Screen for NFAT Activation

In this example, a transcriptional relay system comprising a nucleic acid, as configured in FIGS. 1A and 1B, is used to screen for potential compounds that induce GPCR signaling. For this example, the nucleic acid of FIG. 1A comprises a nuclear factor of activated T-Cell response element (NFAT) activation that results in expression of a synthetic transcription factor Gal4-VPR (comprising Gal4 DNA binding domain and the chimeric activation domain VP64-p65-Rta). The nucleic acid of FIG. 1B comprises a promoter able to be bound and activated by the Gal4-VPR synthetic transcription factor, which results in expression of a reporter element that comprises a luciferase gene and a gene encoding a UMI. The cells used comprise a stably integrated nucleic acid(s) that encodes the system of FIGS. 1A and 1B, and a given GPCR. Each UMI is associated with a given GPCR allowing for CRE expression to be mapped to a particular GPCR. This allows for multiplexing of the assay.

On day 1, plate cells in a 96-well assay plate at 35,000 cells/well in DMEM. On day 2, exchange the media to 0.5% FBS+DMEM. On day 3, remove the media and add a test compound at a desired concentration in 25 uL of Opti-mem. After about 4 hours, remove the media and replace with lysis buffer for RNA extraction. RNA is extracted using standard methods or kits, and subsequently quantified by a standard assay. RNAseq is then performed on an Illumina MiSeq after sequencing library preparation.

### Example 3—Example GPCR Receptor Screen for CRE Activation of Multiple GPCRs

In this example, 100 or more transcriptional relay system comprising nucleic acids, each as configured in FIGS. 1A and 1B, is used to screen for potential compounds that induce GPCR signaling. For this example, each nucleic acid of FIG. 1A comprises a cAMP response element (CRE) activation that results in expression of a synthetic transcription factor Gal4-VPR (comprising Gal4 DNA binding domain and the chimeric activation domain VP64-p65-Rta). Each nucleic acid of FIG. 1B comprises a promoter able to be bound and activated by the Gal4-VPR synthetic transcription factor, which results in expression of a reporter element that comprises a luciferase gene and a gene encoding a UMI. The cell populations used each comprise a stably integrated nucleic acid(s) that encodes the system of FIGS. 1A and 1B, and a given single GPCR. A plurality of 100 or more cell populations, each cell population encoding a single unique GPCR, are mixed together to form a mixed cell population. Each UMI is associated with a given GPCR allowing for CRE expression to be mapped to a particular GPCR. This allows for multiplexing of the assay.

On day 1, plate said mixed cell population in a 96-well assay plate at 35,000 cells/well in DMEM. On day 2, exchange the media to 0.5% FBS+DMEM. On day 3, remove the media and add a test compound at a desired concentration in 25 uL of Opti-mem. After about 4 hours, remove the media and replace with lysis buffer for RNA extraction. RNA is extracted using standard methods or kits, and subsequently quantified by a standard assay. RNAseq is then performed on an Illumina MiSeq after sequencing library preparation.

### Example 4—Amplification of Reporter Output Using a Transcriptional Relay

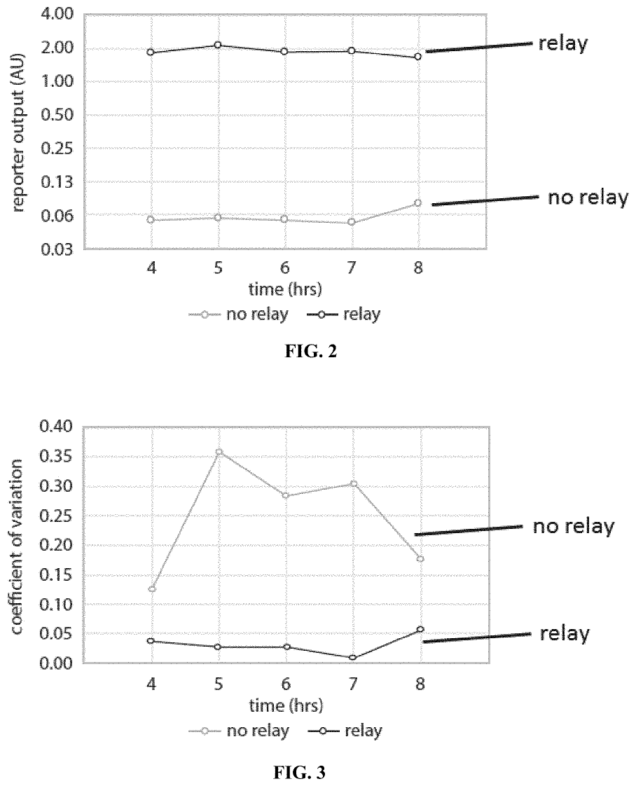

The experiment in this example shows an increase in luciferase signal and a decrease in coefficient of variation of luciferase signal when a transcriptional relay system is used compared to a system without a transcriptional relay. HEK293 derived cells carrying a singly integrated CRE-luciferase or cells carrying a singly integrated UAS-luciferase along with multiple copies of semi-randomly integrated CRE-Gal4-VPR were plated at 30,000 cells/well in a white-walled poly-L-lysine coated 96 well plate in 100 μL DMEM+10% FBS. 50 μL Opti-mem with 45 ng doxycycline was added on top of the cells. 24 hours later, DMSO was added. Cells were treated with DMSO for the indicated periods of time. After the indicated incubation time, the media was aspirated and replaced with 35 μL DMEM and the cells were assayed using the Bright-Glo Luciferase Assay kit [Promega] according to the manufacturer's instructions. The resulting expressed luciferase activity of cells carrying singly integrated CRE-luciferase (gray) and cells carrying a singly integrated UAS-luciferase along with multiple copies of semi-randomly integrated CRE-Gal4-VPR (black) is shown in FIG. 2. The experiment was performed in technical triplicate and the coefficient of variation for each sample was computed in FIG. 3.

### Example 5—Enhancing Fold Induction of the Transcriptional Relay Using a Degron Tag on Gal4-VPR

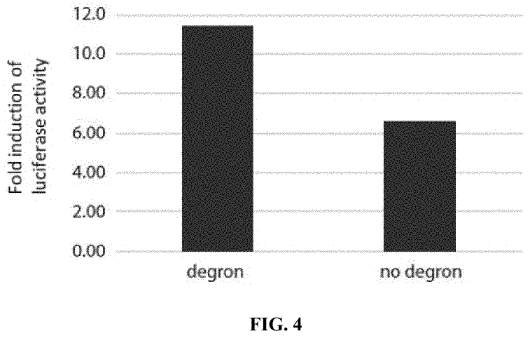

The experiment in this example shows an increase in the fold induction of luciferase signal when a degron tag is included on Gal4-VPR in a transcriptional relay system. HEK293 derived cells carrying a singly-integrated TRE-CHRM3::UAS-luciferase dual gene cassette and multiply semi-randomly integrated FOS-Gal4-VPR-CP (degron) or FOS-Gal4-VPR (no degron) were plated at 30,000 cells/well in a white-walled poly-L-lysine coated 96 well plate in 100 DMEM+10% FBS. 50 μL Opti-mem with 45 ng doxycycline was added on top of the cells. 24 hours later, cells were treated for 8 hours with DMSO or 1 μM carbachol. After the indicated incubation time, the media was aspirated and replaced with 35 μL DMEM and the cells were assayed using the Bright-Glo Luciferase Assay kit [Promega] according to the manufacturer's instructions. The resulting ratio of luciferase activity in carbachol to luciferase activity in DMSO is plotted in FIG. 4.

### Example 6—Cell Lines Comprising NFAT Response Element

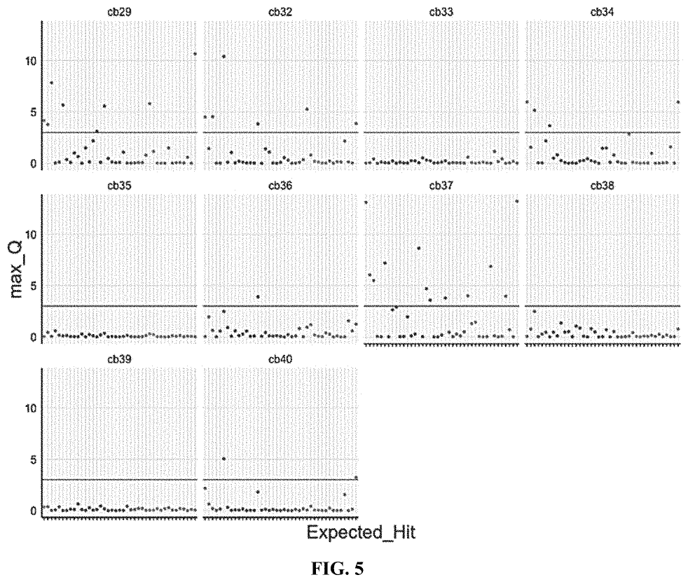

The cell lines described in this example have integrated copies of the NFAT-response element transcriptional relay (NFAT promoter driving transcription of a synthetic transcription factor). These cell lines were generated as a genetically heterogenous pool with respect to copy number and integration site. From this pool, single cell clones were isolated and expanded. These lines were further used to integrate GPCRs and a UAS-Luciferase-barcode reporter to test their ability to detect NFAT signaling in multiplex. From these 10 cell libraries, two were identified that were able to detect the highest number of distinct GPCR hits against control agonists: cb29 (constructed from clone c713) and cb37 (constructed from clone c708) as shown in FIG. 5.

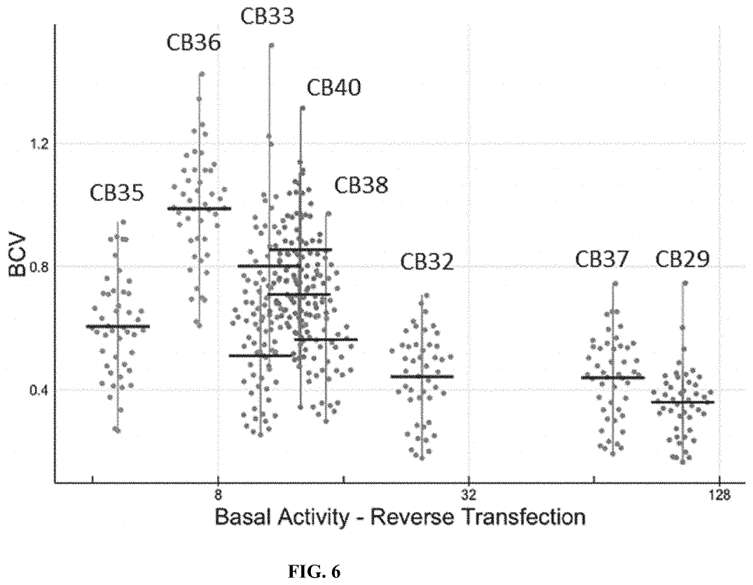

Importantly, it was found that the isoclonal cell lines that gave rise to these two cell libraries shared two common properties. First, these cell lines displayed the highest amount of reporter expression in an unstimulated state (see FIG. 6, “Basal Activity—Reverse Transfection”). Secondly, and likely in a dependent manner, the two corresponding cell libraries showed the lowest level of variation (see FIG. 6, “BCV”).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A transcriptional relay system comprising;
a) a transcription factor nucleic acid comprising a response element regulated promoter nucleotide sequence and a nucleotide sequence encoding a synthetic transcription factor, wherein said response element regulated promoter nucleotide sequence is 5′ to said nucleotide sequence encoding said synthetic transcription factor; and
b) a reporter nucleic acid comprising a synthetic transcription factor promoter nucleotide sequence and a nucleotide sequence encoding a reporter, wherein said synthetic transcription factor promoter nucleotide sequence is 5′ to said nucleotide sequence encoding said reporter, and wherein said synthetic transcription factor promoter nucleotide sequence is able to be bound by said synthetic transcription factor.

2. The transcriptional relay system of claim 1, wherein said response element regulated promoter nucleotide sequence comprises a cAMP response element nucleotide sequence, a NFAT transcription factor response element nucleotide sequence, a FOS promoter nucleotide sequence, or a serum response element nucleotide sequence.

3. The transcriptional relay system of claim 1, wherein said synthetic transcription factor comprises a DNA binding domain from a first transcription factor and a transcription activating domain from a second transcription factor.

4. The transcriptional relay system of claim 3, wherein said DNA binding domain is from Gal4, PPR1, Lac9, or LexA.

5.-8. (canceled)

9. The transcriptional relay system of claim 3, wherein said transcription activating domain comprises VP64, p65, and Rta.

10.-16.

17. The transcriptional relay system of claim 1, wherein said synthetic transcription factor comprises a polypeptide sequence that destabilizes said synthetic transcription factor.

18. The transcriptional relay system of claim 17, wherein said polypeptide sequence that destabilizes said synthetic transcription factor comprises a PEST or a CL1 polypeptide sequence.

19. The transcriptional relay system of claim 1, wherein said synthetic transcription factor promoter nucleotide sequence comprises a nucleotide sequence able to be bound by Gal4, PPR1, Lac9, or LexA.

20. The transcriptional relay system of claim 1, wherein said reporter comprises a fluorescent protein, a luciferase protein, a beta-galactosidase, a beta-glucuronidase, a chloramphenicol acetyltransferase, a secreted placental alkaline phosphatase, or a unique molecular identifier.

21. The transcriptional relay system of claim 20, wherein said reporter comprises a fluorescent protein, a luciferase protein, a beta-galactosidase, a beta-glucuronidase, a chloramphenicol acetyltransferase, or a secreted placental alkaline phosphatase, and a unique molecular identifier.

22. The transcriptional relay system of claim 20, wherein said unique molecular identifier is unique to a test polypeptide, wherein said test polypeptide is encoded by said reporter nucleic acid.

23. The transcriptional relay system of claim 1, wherein said transcription factor nucleic acid comprises a nucleotide sequence proximal to said response element regulated promoter nucleotide sequence that can be bound by said transcriptional repressor.

24. The transcriptional relay system of claim 23, wherein said transcription factor nucleic acid comprises a nucleotide sequence proximal to said response element regulated promoter nucleotide sequence that extends the 5′ untranslated region of an mRNA encoded by said nucleotide sequence encoding said synthetic transcription factor.

25. The transcriptional relay system of claim 24, wherein said 5′ untranslated region of an mRNA encoded by said nucleotide sequence encoding said synthetic transcription factor comprises one or more sequences that reduce translation of said synthetic transcription factor.

26. (canceled)

27. A cell comprising said relay system of claim 1.

28. (canceled)

29. (canceled)

30. The cell of claim 27, wherein the transcription factor nucleic acid, the reporter nucleic acid, or both the transcription factor nucleic acid and the reporter nucleic acid are integrated as a single copy into the genome of the cell.

31.-34. (canceled)

35. The cell of claim 27, wherein the cell or cell population comprises high basal reporter activity.

36. (canceled)

37. The cell or of claim 27, wherein the cell or cell population comprises a low biological coefficient of variance for reporter activity.

38. (canceled)

39. A method for testing an effect of a test agent on the activity of a response element regulated promoter comprising contacting the cell of claim 27 with said test substance.

40. (canceled)
